(19) United States
(12) Patent Application Publication
(10) Pub. No.: US 2012/0203183 A1
(43) Pub. Date: Aug. 9, 2012

(54) SYRINGE

(76) Inventor: Mohsen Tootoonchi, Shiraz (IR)

(21) Appl. No.: 13/020,828

(22) Filed: Feb. 4, 2011

(51) Int. Cl. A61M 5/315; A61M 5/31

(52) U.S. Cl. 604/189; 604/227

(57) ABSTRACT

An improved syringe which prevents the repositioning of a user's hand while administering an injection. The syringe comprises a barrel for holding a fluid medication, a nozzle extending from one end of the barrel, a syringe needle removably fitted to the nozzle, and a plunger slidably disposed within the barrel. The syringe further comprises an elongate handle member disposed substantially parallel to the barrel. The handle member is fixedly connected to the plunger whereby the plunger reciprocates within the barrel as the handle member is subjected to longitudinal to and fro motion.

### SPONSORSHIP STATEMENT

The Iranian National Science Foundation sponsors the present invention for international filing.

The present invention relates to syringes and more particularly to a syringe that does not necessitate a user to reposition his/her grip thereon while operating it.

A typical syringe comprises a barrel, a syringe needle extending from end of the barrel, a plunger slidably disposed within the barrel, and a handle extending from the rear end of the plunger. Due to this location of the handle, a user needs to reposition his/her grip on the syringe when administering an injection or an aspiration to a patient. More particularly, once a proper insertion of the needle into the patient's body is ensured, the user needs to move his/her hand towards the rear end of the syringe in order to operate the handle. Added to this, the user also needs to make use of his/her other hand to ensure a better control over the entire procedure. This is very true especially in the case of infants, children and even animals.

Not only are these conventional syringes unwieldy, and their usage is time consuming, but they also can cause quite discomfort at the receiving end if not handled with ample care and caution. For instance, while repositioning the grip on the syringe, the user must ensure that the syringe doesn't shake, failing which, it can lead to bleeding or even an infection. Also, it is a common occurrence where a user, while administering an injection, tends to accidentally push the needle further while pushing the handle thereby causing pain to a patient.

Therefore, in the light of the above discussion, there is a need for an improved syringe that would reduce the undesirable repositioning of the hands while administering an injection or the like.

### SUMMARY

The present invention presents an improved syringe that overcomes the above-mentioned drawbacks of a conventional syringe. The improved syringe is structurally similar to a conventional one barring the location of a handle member which manipulates the plunger within the syringe barrel. The improved syringe comprises a barrel, a syringe needle extending from one end of the barrel, and a plunger slidably disposed within the barrel. An elongate handle member is extends from the rear end of the plunger and abuts the outer surface of the barrel such that the handle member slides thereover longitudinally.

In order to operate the syringe, a user needs to wrap his/her fingers around the barrel as the thumb rests on the handle member. The plunger within the barrel is manipulated as the handle member is subjected to a longitudinal to and fro motion by the thumb.

This structural arrangement of the syringe, especially the placement of handle member, nullifies the necessity of the user to reposition his/her grip on the syringe while administering an injection or the like. There is no necessity for the user to move his/her hand towards the rear of the syringe in order to actuate the plunger as is the case in the conventional syringes. Now that there is no need for the repositioning, the operation of the syringe is quicker and easier compared to that of the conventional syringes. Also, the user can operate the syringe with a single hand, thereby having better control over the operation procedure.

Other features and advantages of the present invention will be apparent to those skilled in the art from a careful reading of the Detailed Description presented below and accompanied by the drawings.

### BRIEF DESCRIPTION OF THE DRAWINGS

FIG. 1 illustrates a perspective view of a preferred embodiment of the syringe according to the present invention.

FIG. 2 illustrates a front view of the preferred embodiment of the syringe.

FIG. 3 illustrates a perspective view of the barrel of the preferred embodiment.

FIG. 4 illustrates a front view of the barrel of the preferred embodiment.

FIG. 5 illustrates a perspective view of the handle member of the preferred embodiment, the handle member attached to the plunger.

FIG. 6 illustrates a front view of the handle member of the preferred embodiment, the handle member attached to the plunger.

FIG. 7 illustrates a perspective view of an additional embodiment of the syringe according to the present invention.

FIG. 8 illustrates a perspective view of the curved handle member of the additional embodiment, the curved handle member attached to the plunger.

FIG. 9 illustrates a view of the user holding the syringe.

### FIGURES—REFERENCE NUMERALS

10 . . . Preferred embodiment of the syringe
12 . . . Barrel
13 . . . Nozzle
14 . . . Syringe needle
16 . . . Plunger
17 . . . Rear wall
18 . . . Scale
20 . . . Shaft
22 . . . Flange
24 . . . Seal
26 . . . Elongate handle member
28 . . . Elongate rectangular panel
30 . . . Thumb tab
32 . . . Elongate track
34 . . . Longitudinal groove
36 . . . Support tab
38 . . . Additional embodiment of the syringe
40 . . . Curved handle member
42 . . . Curved panel
44 . . . Finger
46 . . . Thumb
48 . . . Fluid medication

### DETAILED DESCRIPTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

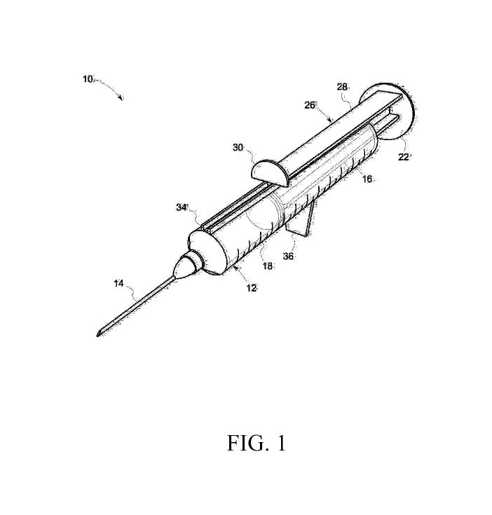

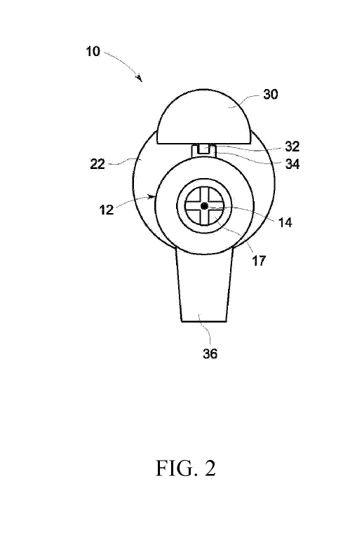

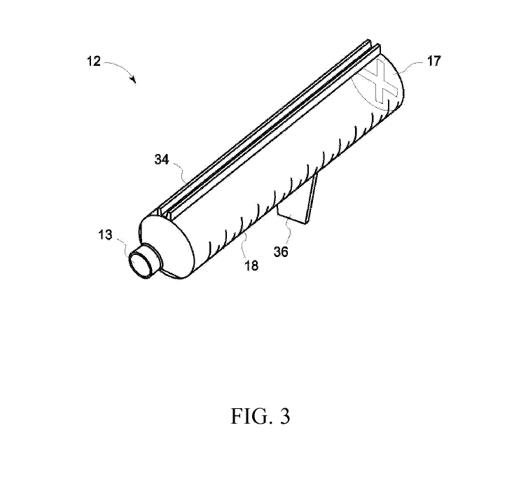

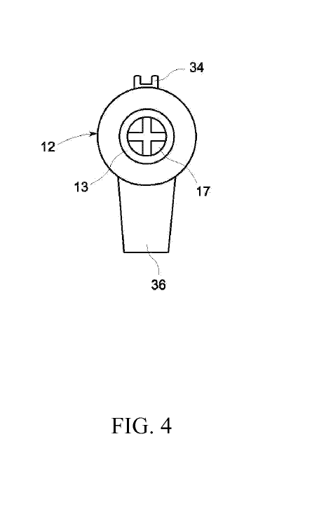

Referring to the drawings, a preferred embodiment of an improved syringe is illustrated and generally indicated as 10 in FIGS. 1 through 6. Referring to FIGS. 1 through 4, the improved syringe 10 comprises a barrel 12, a nozzle 13 extending from one end of the barrel, a syringe needle 14 removably fitted to the nozzle 13, and a plunger 16 disposed within the barrel 12 such that the plunger 16 can freely reciprocate within the barrel 12 along its length. The other end of the barrel 12 terminates in a rear wall 17 facing the nozzle 13, the rear wall 17 having a provision which allows for the reciprocation of the plunger 16. The barrel 12 is essentially of a hollow cylindrical tube made of any translucent material such as glass, plastic, or the like. The barrel 12 includes a longitudinal groove 34 located on its outer surface along its length.

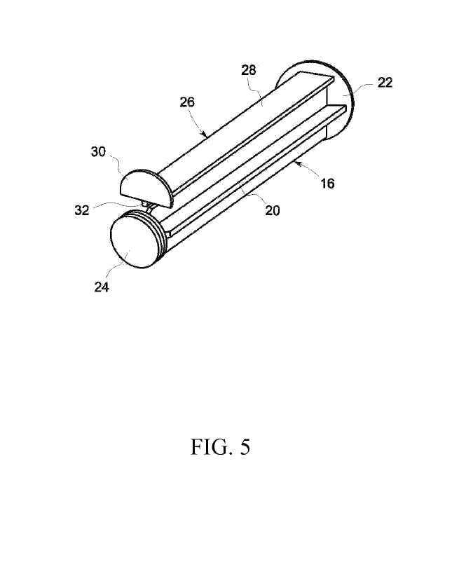

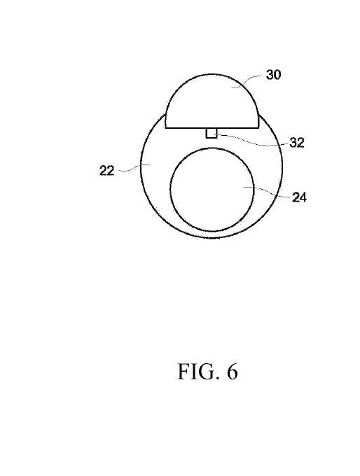

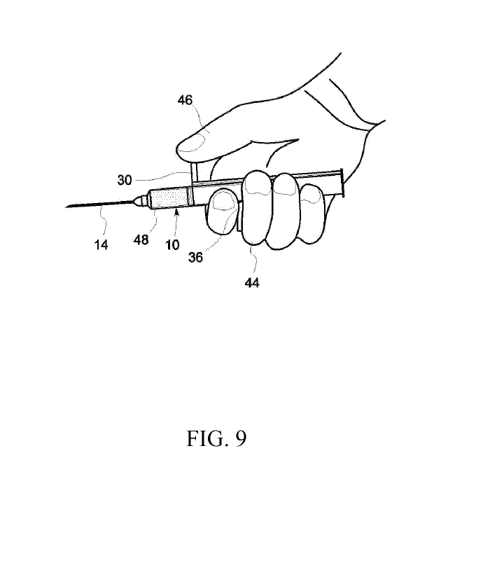

Referring to FIGS. 5 and 6, the plunger 16 comprises a shaft 20, one end of which terminates in a flange 22, while the other is attached to a seal 24. The seal 24 is made of any suitable material such as natural or synthetic rubber, or resilient plastic so as to provide a fluid-tight, frictional engagement with the interior surface of the barrel 12 during its movement therewithin. The barrel further comprises a scale 18 thereon for measuring the volume of fluid medication 48 (FIG. 9) is contained between the seal 20 and the front wall of the barrel 12 from which the nozzle 13 extends.

Referring to FIGS. 1 through 6, the syringe 10 further comprises an elongate handle member 26 fixedly attached to the flange 22 such that the handle member 26 is substantially parallel to the shaft 20 and rests on the outer surface of the barrel 12 abutting the longitudinal groove 34. More particularly, the handle member 26 comprises an elongate rectangular panel 28, the bottom surface of which comprises an elongate track 32 which is slidably received within the longitudinal groove 34 as the handle member 26 rests on the barrel 12. The track 32 is centrally located and runs along the length of the elongate panel 28. One end of the elongate panel 28 is fixedly attached to the flange 22, while the other end comprises a thumb tab 30. The thumb tab 30 is substantially perpendicular to the elongate panel 28 and extends away from the barrel 12. A support tab 36 extends radially from the outer surface of the barrel 12. The support tab 36 is placed diagonally opposite to the handle member 26 and the plane of the support tab 36 is parallel to that of the of thumb tab 30.

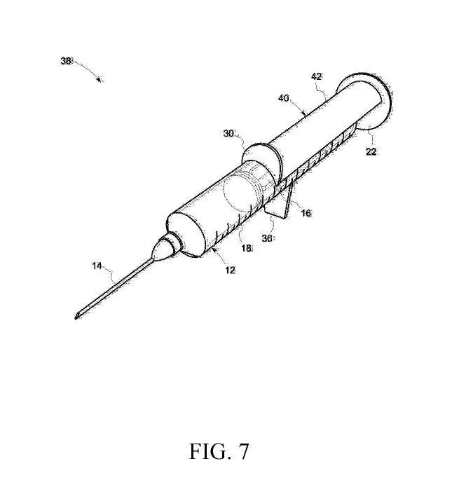

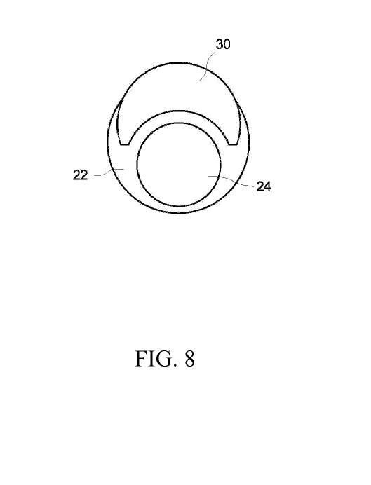

An additional embodiment of an improved syringe is illustrated and generally indicated as 38 in FIGS. 7 and 8. The syringe 38 of the additional embodiment is similar to that of the preferred embodiment except that the handle member 26 of the former is uniformly curved about its longitudinal axis instead. The curved handle member 40 comprises a curved panel 42 and a thumb tab 30 extending from one end thereof, while the other end is fixedly attached the flange 22. The curved panel 42 is same as the elongate panel 28, but uniformly curved about its longitudinal axis. The curved panel 42 conforms to the outer surface of the barrel 12. The syringe 38 omits the longitudinal groove 34 and the elongate track 32 as the curved panel 40 slides on the barrel 12 itself.

Referring to FIG. 9, a user, in order to administer, for instance, an injection, holds the syringe such that his/her fingers 44 wraps around the barrel 12, while his/her thumb 46 rests on the thumb tab 30. The support tab 36 lies between the fingers 44 as user holds the syringe 10. When the user administers an injection to a patient, the user need not reposition his/her hand on the syringe as all it takes is just the movement of the thumb tab 30 with the thumb 46 to discharge the medication 48 through the needle 14.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments, which as a matter of language might be said to fall there between.

What is claimed is:

1. A syringe comprising:
(a) a barrel;
(b) a nozzle extending from one end of the barrel;
(c) a plunger slidably disposed within the barrel; and
(d) an elongate handle member fixedly connected to the plunger such that the handle member is abuts the outer surface of the barrel;
wherein, the longitudinal motion of the handle member causes the plunger to reciprocate relatively within the barrel thereby causing a fluid medication to be drawn into and discharged out of the barrel as the syringe is operated, the medication received and discharged through a syringe needle removably fitted to the nozzle.

2. The syringe of claim 1 comprising a flange extending from the rear end of the plunger, the one end of the handle member fixedly attached to the flange.

3. The syringe of claim 1, wherein the handle member comprises an elongate panel and a thumb tab extending substantially perpendicularly from one end of the elongate panel, the other end of the elongate panel fixedly connected to the plunger, the handle member to be manipulated about the thumb tab by the thumb of a user.

4. The syringe of claim 3, wherein the elongate panel is of a uniform rectangular cross-section.

5. The syringe of claim 3, wherein the bottom surface of the panel conforms closely to the outer surface of the barrel.

6. The syringe of claim 3, wherein the bottom surface of the panel comprises an elongate track slidably received within a longitudinal grove disposed on the outer surface of the barrel.

7. The syringe of claim 1 comprising a support tab extending radially from the outer surface of the barrel, the support tab and the handle member disposed in a diametrically opposing relation, the support tab to be held between two fingers as the handle member is manipulated.

8. The syringe of claim 1, wherein the surface of the barrel comprises a scale.

9. A syringe comprising:
(a) a barrel;
(b) a nozzle extending from one end of the barrel;
(c) a syringe needle removably fitted to the nozzle;
(d) a plunger slidably disposed within the barrel, a fluid medication contained between the nozzle and the plunger; and
(e) an elongate handle member abutting to the outer surface of the barrel, the handle member fixedly connected to the plunger so that the plunger reciprocates within the barrel as the handle member is subjected to longitudinal to and fro motion.

10. The syringe of claim 9 comprising a flange extending from the rear end of the plunger, the handle member fixedly attached to the flange.

11. The syringe of claim 9, wherein the handle member comprises an elongate panel and a thumb tab extending substantially perpendicularly from one end of the panel, the other end of the panel fixedly attached to the plunger, the tab manipulated by the thumb of a user.

12. The syringe of claim 11, wherein the panel is of a uniform rectangular cross-section.

13. The syringe of claim 11, wherein the bottom surface of the panel abuts and conforms to the outer surface of the barrel.

14. The syringe of claim 11, wherein the bottom surface of the panel comprises an elongate track slidably received within a longitudinal grove disposed on the outer surface of the barrel.

15. The syringe of claim 9 comprising a support tab extending radially from the outer surface of the barrel, the support tab and the handle member disposed in a diametrically opposing relation, the support tab to be held between two fingers as the handle member is manipulated.

16. The syringe of claim 1, wherein the surface of the barrel comprises a scale.
